(19) United States
(12) Patent Application Publication
(10) Pub. No.: US 2023/0115562 A1
(43) Pub. Date: Apr. 13, 2023

(54) PROCESS FOR ISOLATING MOLECULES CONTAINED IN THE ORGANO-MINERAL LAYERS OF THE SHELLS OF MARINE BIVALVE MOLLUSCS

(71) Applicant: MBP (MAURITIUS) LTD., Port Louis (MU)

(72) Inventors: Serge CAMPRASSE, Le Mans (FR); Georges CAMPRASSE, Arnage (FR)

(73) Assignee: MBP (MAURITIUS) LTD., Port Louis (MU)

(21) Appl. No.: 17/611,110

(22) PCT Filed: May 12, 2020
(86) PCT No.: PCT/FR2020/050786

§ 371 Date: May 16, 2022

(30) Foreign Application Priority Data
May 13, 2019 (FR) 19 04913

(51) Int. Cl. A61K 35/618; A61K 8/02; A61K 8/04; A61Q 19/08; A61K 8/98

(52) U.S. Cl. CPC A61K 35/618; A61K 8/044; A61K 8/0241; A61K 8/987; A61Q 19/08; A61K 2800/82

(57) ABSTRACT

A process that includes simultaneous and/or sequential steps for separating, extracting and/or isolating all or part of the components contained in the inner aragonitic organo-mineral layer and in the outer calcitic organo-mineral layer of the shells of marine bivalve molluscs. Also, compositions including these components, such as cosmetics and medicinal products.

### FIELD

The invention relates to the implementation of simultaneous and/or sequential steps for separating, extracting and/or isolating all or part of the components contained in the inner aragonitic organo-mineral layer and in the outer calcitic organo-mineral layer of the shells of marine bivalve molluscs such as: Pinctada Maxima- Margaritifera- Martensi- Fucata, as well as Tridacnae Gigas-Maxima- Hippopus Hippopus- Derasa- Tevaroa- Crocea- Squamosa, Porcelanus. The combinations and rearrangements of these extracted components are used in the formulation of medical devices and therapeutically oriented preparations for use in orthopaedic surgery, minimally invasive surgery, stomatology and maxillofacial surgery, dermatology, aesthetic medicine and dermocosmetics.

### BACKGROUND

The structure and the physico-chemical composition of the inner aragonitic organo-mineral layer of the shell of the above-mentioned molluscs contain two fractions: a mineral fraction consisting of aragonite biocrystals, a metastable, polymorphic, biogenic form of calcium carbonate CaCO₃, crystallising in the orthorhombic system, and an organic fraction consisting mainly of protein and non-protein components, pigments (melanin, beta-carotene, etc.), fatty acids and total lipids, especially polyunsaturated.

The outer calcitic organo-mineral layer of the shell of the same molluscs also consists of a mineral fraction, composed of prisms of calcite, another polymorphic form of calcium carbonate, crystallising in the rhombohedral system, and an organic fraction also consisting of soluble and insoluble protein and non-protein components, pigments and metals. The outer calcitic organo-mineral layer is richer than the inner aragonitic organo-mineral layer in melanic pigments and metals associated with porphyrins and enzymes.

Generally, the active molecules contained in the organic fractions of the inner aragonitic and outer calcitic organo-mineral layers of the shells of the above-mentioned molluscs are protein and non-protein components extracted by cold hydrolysis. These components are soluble and insoluble biopolymers which include proteins, polypeptides and polysaccharides, as well as bio-monomers, amino acids and monosaccharides. All of them have numerous biological and pharmacological, osteo-inductive and healing properties, among others. These properties are due to the presence of glycoproteins related to growth factors. However, the processes conventionally used to extract these molecules do not make it possible to extract all the molecules, especially those of low-molecular-weight with properties of interest, mainly antibiomimetics such as glycosamines, as well as lipids and polyunsaturated fatty acids, without forgetting pigments, metalloenzymes and metalloporphyrins. It is known that the organic fraction of the inner aragonitic organo-mineral layer of the shell of the molluscs mentioned contains, inter alia, fatty acids and total lipids (palmitic acid, stearic acid); it is the natural marine biomaterial richest in polyunsaturated fatty acids, in proportions of 0.2% to 3%. These are mostly polar and apolar compounds represented by hydroxylated and non-hydroxylated ceramides, cholesterol sulphate and/or acetate, triglycerides and omega-3s.

When it is known that fatty acids have anti-inflammatory and immune properties, that they seem to play an inhibitory role in the development of certain tumour processes, in rheumatoid arthritis and in autoimmune diseases, and that these same lipids and fatty acids induce an overexpression of filaggrin, protein of the superficial layer of the skin which has membrane transglutaminase inhibitory properties (set of insoluble proteinic polymers involved in certain dermatoses), their advantage in the formulation of preparations for therapeutic purposes is therefore understandable.

### Technical Problem

For this reason, there is a need for a process to optimise the isolation of molecules contained in the inner aragonitic and outer calcitic organo-mineral layers of the shells of marine bivalve molluscs.

It is to the inventors’ credit that they have responded to this need with a process that can implement a succession of mechanical, acoustic, physical and chemical steps in order to optimise the separation, purification and physico-chemical reactivity of the molecules contained in the inner aragonitic and outer calcitic organo-mineral layers of the shells, and to potentiate their properties.

### SUMMARY

Therefore, a first subject matter of the invention is a process for isolating molecules contained in an aragonitic organo-mineral layer and/or in a calcitic organo-mineral layer of a shell of a marine bivalve mollusc comprising the following steps:
(a) Grinding the aragonitic organo-mineral layer and/or the calcitic organo-mineral layer to obtain an aragonitic powder and/or a calcitic powder;
(b) Hot percolating the aragonitic powder and/or the calcitic powder to obtain:
   on the one hand, a saturated aragonitic solution and/or a saturated calcitic solution, said saturated aragonitic solution comprising an aragonitic liquid phase and an aragonitic solid phase and said saturated calcitic solution comprising a calcitic liquid phase and a calcitic solid phase, and
   on the other hand, an aragonitic percolation powder and/or a calcitic percolation powder;
(c) Separating the saturated aragonitic solution and/or the saturated calcitic solution to recover:
   on the one hand, the aragonitic liquid phase and/or the calcitic liquid phase, and
   on the other hand, the aragonitic solid phase and/or the calcitic solid phase; and
(d) Treating the aragonitic percolation powder and/or the calcitic percolation powder with supercritical CO₂ to obtain:
   on the one hand, a supercritical CO₂-treated aragonitic powder and/or a supercritical CO₂-treated calcitic powder, and
   on the other hand, all or part of the soluble molecules contained in the aragonitic organo-mineral layer and/or in the calcitic organo-mineral layer.

For the purposes of the present invention, “supercritical CO₂-treated aragonitic powder” means a powder from which all or some of the soluble molecules contained in the aragonitic organo-mineral layer have been removed.

For the purposes of the present invention, “supercritical CO₂-treated calcitic powder” means a powder from which all or some of the soluble molecules contained in the calcitic organo-mineral layer have been removed.

Depending on the method of production, the marine bivalve mollusc can be selected from Pinctada Maxima, Pinctada Margaritifera, Pinctada Martensi, Pinctada Fucata, Tridacnae Gigas, Tridacnae Maxima, Tridacnae Hippopus Hippopus, Tridacnae Derasa, Tridacnae Tevaroa, Tridacnae Crocea, Tridacnae Squamosa, Tridacnae Porcelanus and mixtures thereof.

According to an embodiment, the shell may undergo, before the grinding step (a), a milling step in order to obtain, on the one hand, the aragonitic organo-mineral layer and, on the other hand, the calcitic organo-mineral layer, said milling step being optionally preceded by a step of pre-treating the shell selected from cleaning, ultrasonic treatment, rinsing, sterilisation, drying, immersion in an isotonic bath and mixtures thereof.

According to an embodiment, the calcitic organo-mineral layer obtained in the milling step and/or used in the grinding step (a) may be powdery and have a particle size between 2 mm and 500 µm.

According to a particular embodiment, the grinding step (a) can be carried out by planetary grinding.

According to a very particular embodiment, the planetary grinding of step (a) may comprise one or more cycles, in particular two cycles. Each planetary grinding cycle may, for example, be carried out by a dry or wet method, in particular the first grinding cycle may be carried out by a dry method and the second cycle may be carried out by a wet method.

According to an embodiment, the grinding step (a) can be carried out by:
crushing the aragonitic organo-mineral layer and/or the calcitic organo-mineral layer to obtain a crushed aragonitic powder and/or a crushed calcitic powder, then
grinding the crushed aragonitic powder and/or the crushed calcitic powder to obtain the aragonitic powder and/or the calcitic powder.

According to a particular embodiment, the grinding of crushed aragonitic powder and/or crushed calcitic powder can be carried out by planetary grinding as described above.

According to an embodiment, the crushed aragonitic powder and/or the crushed calcitic powder may have a particle size between 10 µm and 2 mm.

According to an embodiment, the aragonitic powder and/or calcitic powder obtained in the grinding step (a) may have a particle size of 50 nm to 300 µm.

According to an embodiment, the hot percolation step (b) may be carried out by wet sieving with a liquid having a temperature above 30° C., in particular from 35° C. to 75° C., especially from 40° C. to 50° C.

According to an embodiment, the liquid used in the hot percolation step (b) may be an aqueous solution, in particular an aqueous solution comprising methanol, an aqueous solution comprising a urea solution or a mixture thereof.

According to a particular embodiment, the aqueous solution may contain from 1% to 10% methanol, in particular from 2% to 7% methanol, more particularly from 4.5% to 5.5% methanol.

According to a particular embodiment, the hot percolation step (b) can be carried out with a sieve shaker comprising:
a cover fitted with an opening to receive a pipe for the liquid inlet,
a plurality of sieves for collecting the aragonitic percolation powder and/or the calcitic percolation powder, in particular 2 to 10 sieves, more particularly 5 to 7 sieves, even more particularly 6 sieves, and the pore diameter of the pores of two successive sieves in the direction of flow of the liquid decreases,
a collection bottom fitted with a conduit to collect the saturated aragonitic solution and/or the saturated calcitic solution.

According to a very particular embodiment, the sieve shaker may comprise 6 sieves with pore diameters of, in the direction of liquid flow, 315 µm, 250 µm, 125 µm, 45 µm, 20 µm and 10 µm.

According to an embodiment, the aragonitic percolation powder and/or the calcitic percolation powder may have a particle size larger than the diameter of the sieve with the smallest diameter among the sieve diameters of the sieve shaker, in particular a particle size larger than 10 µm, more particularly a particle size of 10 µm to 300 µm.

According to an embodiment, the aragonitic liquid phase of the saturated aragonitic solution may comprise water-soluble and fat-soluble molecules contained in the aragonitic organo-mineral layer.

According to an embodiment, the aragonitic solid phase of the saturated aragonitic solution may comprise:
the insoluble molecules contained in the aragonitic organo-mineral layer, such as insoluble protein and non-protein molecules, and
a powder with a particle size less than or equal to the diameter of the sieve whose diameter is the smallest among the sieve diameters of the sieve shaker, in particular with a particle size less than or equal to 10 µm, in particular from 50 nm to 10 µm.

According to an embodiment, the calcitic liquid phase of the saturated calcitic solution may comprise water-soluble and fat-soluble molecules contained in the calcitic organo-mineral layer.

According to an embodiment, the calcitic solid phase of the saturated calcitic solution may comprise:
the insoluble molecules contained in the calcitic organo-mineral layer, such as insoluble protein and non-protein molecules, and
a powder whose particle size is less than or equal to the diameter of the sieve whose diameter is the smallest among the diameters of the sieves of the sieve shaker, in particular whose particle size is less than or equal to 10 µm, in particular from 50 nm to 10 µm.

According to a preferred embodiment of implementation, separation step (c) is carried out by centrifugation and the recovered liquid phase is referred to as supernatant and the recovered solid phase is referred to as pellet.

According to an embodiment, the aragonitic pellet powder and/or the calcitic pellet powder, in particular the aragonitic pellet powder, may undergo a spheronisation step.

According to an embodiment, the aragonitic pellet and/or the calcitic pellet, in particular the calcitic pellet, may undergo the supercritical CO₂ treatment step (d).

According to an embodiment, the supercritical CO₂ treatment step (d) can be implemented in an installation comprising:
an inlet and storage tank for CO₂ in a gaseous state,
a condenser for the transformation of gaseous CO₂ into liquid CO₂,
a liquid CO₂ storage tank,
a heat exchanger for the transformation of liquid CO₂ into supercritical CO₂,
a reactor where the extraction of soluble molecules takes place, and
one or more extractors.

The soluble molecules obtained in the supercritical CO₂ treatment step (d) are at least those among soluble biopolymers, fatty acids, lipids, soluble pigments and their mixture.

Soluble fatty acids, lipids and pigments cannot be obtained by the soluble biopolymer extraction process disclosed in FR 3 037 801 filed by the inventors of the present patent application.

According to a particular embodiment, the particle size of the supercritical CO₂-treated aragonitic powder is less than or equal to the particle size of the aragonitic percolation powder, in particular equal to the particle size of the aragonitic percolation powder.

According to a particular embodiment, the particle size of the supercritical CO₂-treated calcitic powder is less than or equal to the particle size of the calcitic percolation powder, in particular equal to the particle size of the calcitic percolation powder.

According to a particular embodiment, the isolation process may comprise, after the separation step (c) carried out by centrifugation, the following steps:
(e) Filtering the aragonitic supernatant and/or the calcitic supernatant to obtain a filtered aragonitic supernatant and/or a filtered calcitic supernatant;
(f) Concentrating the filtered aragonitic supernatant and/or the filtered calcitic supernatant to obtain an aragonitic concentrate and/or a calcitic concentrate;
(g) Sonicating the aragonitic concentrate and/or the calcitic concentrate to obtain an aragonitic colloidal emulsion and/or a calcitic colloidal emulsion.

According to an embodiment, the filtration step (e) can be carried out on a Celite bed or a membrane.

According to an embodiment, the aragonitic concentrate and/or the calcitic concentrate may comprise at least one metal selected from Mn, Fe, Zn, Ba, Sr, Mg, Cu, Al, Ni, V, Cr, Mo and mixtures thereof.

According to an embodiment, the sonication step (g) can be carried out with a sonotrode at a frequency between 20 kHz and 200 kHz.

According to an embodiment, the process may comprise, after the supercritical CO₂ treatment step (d), the following steps:
(h) Cold acid hydrolysis of the supercritical CO₂-treated aragonitic powder and/or the supercritical CO₂-treated calcitic powder to extract insoluble molecules present in said powders; and
(i) Washing and supercentrifugation to isolate and recover said insoluble molecules.

The insoluble molecules recovered during the steps of cold acid hydrolysis (h) and washing and supercentrifugation (i) are at least those from insoluble biopolymers, insoluble organic pigments and mixtures thereof.

According to an embodiment, the cold acid hydrolysis step (h) can be carried out:
at a temperature below 10° C., in particular below 5° C., more particularly between 1 and 4° C., and
with an aqueous solution comprising acetic acid and whose pH is acidic, in particular below 6, more particularly below 4.5.

According to a particular embodiment, the steps of cold acid hydrolysis (h) and washing and supercentrifugation (i) may be carried out once or several times.

According to an embodiment, the insoluble molecules recovered can then be dried to obtain a dry extract of insoluble molecules.

According to a very particular embodiment, the grinding step (a) is carried out separately on the aragonitic organo-mineral layer and on the calcitic organo-mineral layer to obtain the aragonitic powder and the calcitic powder.

According to a very particular embodiment, the hot percolation step (b) is carried out separately on the aragonitic powder and on the calcitic powder to obtain:
on the one hand, the saturated aragonitic solution, and, on the other hand, the aragonitic percolation powder and
on the one hand, the saturated calcitic solution, and, on the other hand, the calcitic percolation powder.

According to a very particular embodiment, the separation step (c) is carried out separately on the saturated aragonitic solution and the saturated calcitic solution in order to recover:
on the one hand, the aragonitic liquid phase, and, on the other hand, the aragonitic solid phase, and
on the one hand, the calcitic liquid phase, and, on the other hand, the calcitic solid phase.

According to a very particular embodiment, the separation step (c) is carried out by centrifugation and separately on the saturated aragonitic solution and the saturated calcitic solution in order to recover:
on the one hand, the aragonitic supernatant, and, on the other hand, the aragonitic pellet, and
on the one hand, the calcitic supernatant, and, on the one hand, the calcitic pellet.

According to a very particular embodiment, the supercritical CO₂ treatment step (d) is carried out separately on the aragonitic percolation powder and on a mixture of the calcitic percolation powder and the calcitic pellet.

According to a specific embodiment:
the grinding step (a) is carried out separately on the aragonitic organo-mineral layer and on the calcitic organo-mineral layer in order to obtain the aragonitic powder and the calcitic powder;
the hot percolation step (b) is carried out separately on the aragonitic powder and on the calcitic powder in order to obtain:
   on the one hand, the saturated aragonitic solution, and, on the other hand, the aragonitic percolation powder, and
   on the one hand, the saturated calcitic solution, and, on the other hand, the calcitic percolation powder;
the separation step (c) is carried out by centrifugation and separately on the saturated aragonitic solution and the saturated calcitic solution in order to recover:
   on the one hand, the aragonitic supernatant, and, on the other hand, the aragonitic pellet, and
   on the one hand, the calcitic supernatant, and, on the other hand, the calcitic pellet; and
the supercritical CO₂ treatment step (d) is carried out separately on the aragonitic percolation powder and on a mixture of the calcitic percolation powder and the calcitic pellet.

According to a very particular embodiment, the filtration step (e) is carried out separately on the aragonitic supernatant and on the calcitic supernatant in order to obtain the filtered aragonitic supernatant and the filtered calcitic supernatant.

According to a very particular embodiment, the concentration step (f) is carried out on a mixture of the filtered aragonitic supernatant and the filtered calcitic supernatant to obtain a mixture of concentrates.

According to a very particular embodiment, the sonication step (g) is carried out on a mixture of concentrates to obtain a mixture of colloidal emulsions.

According to a very particular embodiment, the cold acid hydrolysis step (h) is carried out on a mixture of supercritical CO₂-treated aragonitic powder and supercritical CO₂-treated calcitic powder in order to extract the insoluble molecules from the mixture of said powders.

According to a very particular embodiment, the washing and supercentrifugation step (i) is carried out to isolate and recover the insoluble molecules extracted from the mixture of supercritical CO₂-treated aragonitic powder and CO₂-treated calcitic powder during the cold acid hydrolysis step (h).

According to a specific embodiment:
the filtration step (e) is carried out separately on the aragonitic supernatant and on the filtered calcitic supernatant in order to obtain the filtered aragonitic supernatant and the filtered calcitic supernatant;
the concentration step (f) is performed on a mixture of the filtered aragonitic supernatant and the filtered calcitic supernatant to obtain a mixture of concentrates;
the sonication step (g) is carried out on a mixture of concentrates to obtain a mixture of colloidal emulsions;
the cold acid hydrolysis step (h) is carried out on a mixture of supercritical CO₂-treated aragonitic powder and supercritical CO₂-treated calcitic powder to extract the insoluble molecules from the mixture of said powders; and
the washing and supercentrifugation step (i) is carried out to isolate and recover the insoluble molecules extracted from the mixture of supercritical CO₂-treated aragonitic powder and CO₂-treated calcitic powder in the cold acid hydrolysis step (h).

According to a preferred embodiment:
the grinding step (a) is carried out separately on the aragonitic organo-mineral layer and on the calcitic organo-mineral layer in order to obtain the aragonitic powder and the calcitic powder;
the hot percolation step (b) is carried out separately on the aragonitic powder and on the calcitic powder in order to obtain:
   on the one hand, the saturated aragonitic solution and, on the other hand, the aragonitic percolation powder, and
   on the one hand, the saturated calcitic solution a and, on the other hand, the calcitic percolation powder;
the separation step (c) is carried out by centrifugation and separately on the saturated aragonitic solution and the saturated calcitic solution in order to recover:
   on the one hand, the aragonitic supernatant, and, on the other hand, the aragonitic pellet, and
   on the one hand, the calcitic supernatant, and, on the other hand, the calcitic pellet;
the supercritical CO₂ treatment step (d) is carried out separately on the aragonitic percolation powder and on a mixture of the calcitic percolation powder and the calcitic pellet;
the filtration step (e) is carried out separately on the aragonitic supernatant and on the filtered calcitic supernatant to obtain the filtered aragonitic supernatant and the filtered calcitic supernatant;
the concentration step (f) is carried out on a mixture of the filtered aragonitic supernatant and the filtered calcitic supernatant to obtain a mixture of concentrates;
the sonication step (g) is carried out on a mixture of concentrates to obtain a mixture of colloidal emulsions;
the cold acid hydrolysis step (h) is carried out on a mixture of supercritical CO₂-treated aragonitic powder and supercritical CO₂-treated calcitic powder to extract the insoluble molecules from the mixture of said powders; and
the washing and supercentrifugation step (i) is carried out to isolate and recover the insoluble molecules extracted from the mixture of supercritical CO₂-treated aragonitic powder and CO₂-treated calcitic powder in the cold acid hydrolysis step (h).

According to a particular embodiment, the isolation process of the invention makes it possible to obtain:
the aragonitic solid phase and/or the calcitic solid phase recovered during the separation step (c), said solid phases possibly being spheronised,
the soluble molecules obtained in the supercritical CO₂ treatment step (d), in particular at least those among soluble biopolymers, fatty acids, lipids, soluble pigments and mixtures thereof,
the aragonitic colloidal emulsion and/or the calcitic colloidal emulsion obtained in the sonication step (g), or
the insoluble molecules recovered during the cold acid hydrolysis (h) and washing and supercentrifugation (i) steps, in particular at least those from soluble biopolymers, fatty acids, lipids, soluble pigments and mixtures thereof.

According to a very particular embodiment, the isolation process of the invention makes it possible to obtain:
the aragonitic solid phase recovered during the separation step (c), said aragonitic solid phase being optionally spheronised,
the mixture of colloidal emulsions obtained in step (g),
the soluble molecules obtained in the supercritical CO₂ treatment step (d), in particular at least those among soluble biopolymers, fatty acids, lipids, soluble pigments and mixtures thereof, and
the insoluble molecules recovered during the cold acid hydrolysis (h) and washing and supercentrifugation (i) steps, in particular at least those from soluble biopolymers, fatty acids, lipids, soluble pigments and mixtures thereof.

When the separation step (c) is carried out by centrifugation, then the isolation process of the invention makes it possible to obtain the aragonitic pellet in place of the aragonitic solid phase and/or the calcitic pellet in place of the calcitic solid phase, said pellets being optionally spheronised.

Another subject matter of the invention is a composition comprising:
the aragonitic solid phase and/or the calcitic solid phase recovered during the separation step (c) as described above in connection with the isolation process, said solid phases optionally being spheronised, and at least one component selected from:
the soluble molecules obtained in the supercritical CO₂ treatment step (d) as described above in connection with the isolation process, in particular at least those from soluble biopolymers, fatty acids, lipids, soluble pigments and mixtures thereof,
the aragonitic and/or calcitic colloidal emulsion obtained in the sonication step (g) as described above in connection with the isolation process, and
the insoluble molecules recovered during the cold acid hydrolysis (h) and washing and supercentrifugation (i) steps, in particular at least those from soluble biopolymers, fatty acids, lipids, soluble pigments and mixtures thereof.

According to a particular embodiment, the composition may comprise:
the aragonitic solid phase recovered during the separation step (c) as described above in connection with the isolation process, said aragonitic solid phase being optionally spheronised, and at least one component selected from:
the soluble molecules obtained in the supercritical CO₂ treatment step (d) as described above in connection with the isolation process, in particular at least those from soluble biopolymers, fatty acids, lipids, soluble pigments and mixtures thereof,
the mixture of colloidal emulsions obtained in the sonication step (g) as described above in connection with the isolation process,
the insoluble molecules recovered during the cold acid hydrolysis (h) and washing and supercentrifugation (i) steps, in particular at least those from soluble biopolymers, fatty acids, lipids, soluble pigments and mixtures thereof.

According to an embodiment, the composition may result from the mixing of these compounds.

According to an embodiment, the composition may also include a mixture of essential and vegetable oils.

According to a particular embodiment, the composition comprises:
the aragonitic solid phase and/or the calcitic solid phase recovered during the separation step (c) as described above in connection with the isolation process, said solid phases optionally being spheronised,
the aragonitic and/or calcitic colloidal emulsion obtained in the sonication step (g) as described above in connection with the isolation process.

According to a very particular embodiment, the composition may comprise:
the aragonitic solid phase recovered during the separation step (c) as described above in connection with the isolation process, said aragonitic solid phase being optionally spheronised, and
the mixture of colloidal emulsions obtained in the sonication step (g) as described above in connection with the isolation process.

According to a particular embodiment, the composition may comprise:
the aragonitic solid phase and/or the calcitic solid phase recovered during the separation step (c) as described above in connection with the isolation process, said solid phases optionally being spheronised,
the soluble molecules obtained in the supercritical CO₂ treatment step (d) as described above in connection with the isolation process, in particular at least those from soluble biopolymers, fatty acids, lipids, soluble pigments and mixtures thereof, and
the aragonitic and/or calcitic colloidal emulsion obtained in the sonication step (g) as described above in connection with the isolation process.

According to a very particular embodiment, the composition may comprise:
the aragonitic solid phase recovered during the separation step (c) as described above in connection with the isolation process, said aragonitic solid phase being optionally spheronised,
the soluble molecules obtained in the supercritical CO₂ treatment step (d) as described above in connection with the isolation process, in particular at least those among soluble biopolymers, fatty acids, lipids, soluble pigments and mixtures thereof, and
the mixture of colloidal emulsions obtained in the sonication step (g) as described above in connection with the isolation process.

According to a particular embodiment, the composition may comprise:
the aragonitic solid phase and/or the calcitic solid phase recovered during the separation step (c) as described above in connection with the isolation process, said solid phases optionally being spheronised,
the soluble molecules obtained in the supercritical CO₂ treatment step (d) as described above in connection with the isolation process, in particular at least those among soluble biopolymers, fatty acids, lipids, soluble pigments and mixtures thereof,
the aragonitic and/or calcitic colloidal emulsion obtained in the sonication step (g) as described above in connection with the isolation process, and
the insoluble molecules recovered during the cold acid hydrolysis (h) and washing and supercentrifugation (i) steps, in particular at least those from soluble biopolymers, fatty acids, lipids, soluble pigments and mixtures thereof.

According to a very particular embodiment, the composition may comprise:
the aragonitic solid phase recovered during the separation step (c) as described above in connection with the isolation process, said aragonitic solid phase being optionally spheronised,
the soluble molecules obtained in the supercritical CO₂ treatment step (d) as described above in connection with the isolation process, in particular at least those among soluble biopolymers, fatty acids, lipids, soluble pigments and mixtures thereof,
the mixture of colloidal emulsions obtained in the sonication step (g) as described above in connection with the isolation process, and
the insoluble molecules recovered during the cold acid hydrolysis (h) and washing and supercentrifugation (i) steps, in particular at least those from soluble biopolymers, fatty acids, lipids, soluble pigments and mixtures thereof.

According to a specific embodiment, the aragonitic solid phase recovered during the separation step (c) included in the composition concerned by the invention may be replaced by the aragonitic pellet recovered during the separation step (c) carried out by centrifugation, said aragonitic pellet optionally being spheronised.

According to a specific embodiment, the calcitic solid phase recovered during the separation step (c) included in the composition concerned by the invention may be replaced by the calcitic pellet recovered during the separation step (c) carried out by centrifugation as described above in connection with the isolation process, said calcitic pellet optionally being spheronised.

Another subject matter of the invention is a composition as described above for use as a medicinal product.

Another subject matter of the invention is a method of therapeutic treatment in which the composition as described above is administered to a subject in need thereof.

According to an embodiment, the therapeutic treatment is selected from the treatment of a skin disease, the prevention of a skin disease.

According to an embodiment, the skin disease is selected from dermatitis, dermatoses such as vitiligo and psoriasis.

According to an embodiment, the composition can be administered topically.

According to another embodiment, the composition can also be used as bone substitute, cement, implant, osteosynthesis devices, medical device in therapy.

According to an embodiment, the bone substitute may be selected from an extrudable bone substitute, in particular packaged in a vacuum syringe, a bone substitute with a porous collagen support, a bone substitute with a mineral screen of animal or human origin and mixtures thereof.

According to an embodiment, the cement is selected from stent cement, injectable cement for minimally invasive surgery in vertebroplasty and kyphoplasty.

Another subject matter of the invention is a non-therapeutic use of a composition as described above.

Another subject matter of the invention is a method of non-therapeutic treatment in which the composition as described above is applied to a person in need thereof.

According to an embodiment, the composition can be used in cosmetics, especially in the treatment of ptosis, dermocutaneous depressions, deep and superficial wrinkles, prevention of body ageing.

Another subject matter of the invention is the use of the composition as described above as culture medium, in particular as culture medium for the maturation and/or proliferation of stem cells or progenitor cells.

According to an embodiment, the composition used as culture medium may comprise:
the aragonitic solid phase and/or the calcitic solid phase recovered during the separation step (c) as described above in connection with the isolation process, said solid phases optionally being spheronised,
the soluble molecules obtained in the supercritical CO₂ treatment step (d) as described above in connection with the isolation process, in particular at least those among soluble biopolymers, fatty acids, lipids, soluble pigments and mixtures thereof, and
the aragonitic and/or calcitic colloidal emulsion obtained in the sonication step (g) as described above in connection with the isolation process.

According to an embodiment, the composition used as culture medium may comprise:
the aragonitic pellet and/or the calcitic pellet recovered during the separation step (c) carried out by centrifugation as described above in connection with the isolation process, said pellets optionally being spheronised,
the soluble molecules obtained in the supercritical CO₂ treatment step (d) as described above in connection with the isolation process, in particular at least those among soluble biopolymers, fatty acids, lipids, soluble pigments and mixtures thereof, and
the aragonitic and/or calcitic colloidal emulsion obtained in the sonication step (g) as described above in connection with the isolation process.

Another subject matter of the invention is another composition comprising:
the soluble molecules obtained in the supercritical CO₂ treatment step (d) as described above in connection with the isolation process, in particular at least those among soluble biopolymers, fatty acids, lipids, soluble pigments and mixtures thereof,
the aragonitic and/or calcitic colloidal emulsion obtained in the sonication step (g) as described above in connection with the isolation process.

According to a particular embodiment, the other composition may comprise:
the soluble molecules obtained in the supercritical CO₂ treatment step (d) as described above in connection with the isolation process, in particular at least those from soluble biopolymers, fatty acids, lipids, soluble pigments and mixtures thereof,
the mixture of colloidal emulsions obtained in the sonication step (g) as described above in connection with the isolation process.

Another subject matter of the invention is the other composition as described above for use as a medicinal product.

Another subject matter of the invention is a method of therapeutic treatment in which the other composition as described above is administered to a subject in need thereof.

According to an embodiment, the therapeutic treatment is selected from chronic autoimmune pathologies.

According to an embodiment, the chronic autoimmune pathologies may be rheumatoid arthritis, Crohn’s disease, arteriosclerosis, type II diabetes, ankylosing spondylitis, ulcerative colitis, psoriasis and psoriatic arthritis, particularly psoriasis.

According to an embodiment, the other composition may be administered by intramuscular, intravenous and/or subcutaneous injection.

### DETAILED DESCRIPTION

The invention consists of a process implementing a hot percolation step (b) of aragonitic and calcitic powders, i.e. the inner aragonitic and outer calcitic organo-mineral layers of the shells reduced to powder during a grinding step (a). The saturated solution resulting from the hot percolation step (b) may then undergo a separation step (c) carried out by centrifugation, followed optionally by a concentration step (f) and a sonication step (g). All or part of the powder which has been the subject of the hot percolation step (b) undergoes a supercritical CO₂ treatment step (d). The aragonitic and calcitic powders set aside during the hot percolation (b) and supercritical CO₂ treatment (d) steps, as well as the powders resulting from the separation step (c) carried out by centrifuging the liquid solution resulting from the hot percolation step (b) of the aragonitic organo-mineral layer and/or the calcitic organo-mineral layer (hereafter aragonitic and/or calcitic pellet), may undergo cold acid hydrolysis (step (h)).

### Pre-Treatment of Shells

The shells of the molluscs concerned, after being cleaned, are subjected to ultrasonic treatment for, for example, 30 minutes in a solution of mains water at 50° C. with a bactericidal, disinfectant, virucidal preparation of the UC38 type. The shells thus treated are then rinsed, for example, with mains water at a temperature of approximately 50° C., then immersed in a 2.5% stabilised sodium hypochlorite solution for 30 minutes, rinsed with mains water for 5 minutes. They are then immersed in a surgical Calbenium® solution for 1 hour, dried by a stream of air, then packaged in autoclavable bags.

The shells can then undergo one or more sterilisation steps. The sterilisation step may consist of three successive “medical prion” sterilisations at 132° C. for 85 min each. The sterilised shells can then be dried in a stream of air and set aside.

The shells can be immersed in a bath of isotonic “marine plasma”. This step can advantageously re-equilibrate the initial water and mineral content of the aragonitic and calcitic organo-mineral layers of the shells, which may have been modified if the shells were out of the marine environment for too long and/or underwent successive treatments. This immersion step may last up to 48 hours. For example, the mineral content in isotonic “marine plasma” may be as follows: Na 12.88 mg/L, Br 66.3 mg/L, Zn 0.083 mg/L, K 493 mg/L, P 0.707 mg/L, Ca 442 mg/L, Mg 1.29 mg/L, Cu 0.007 mg/L. The shells are then air-dried and set aside.

### Milling of the Shells, Crushing and Grinding Step (a)

In order to treat the aragonitic organo-mineral layer and the calcitic organo-mineral layer of the shells separately, the calcitic organo-mineral layer may undergo a milling step. This milling step can be carried out using a coarse-grained diamond milling wheel, for example, under a current of filtered and cooled seawater at a temperature between 2 and 4° C. A powdery milling product with a grain size of 2 mm to 500 µm is then obtained. The milling product is set aside together with the aragonitic organo-mineral layer, which has been freed of the calcitic organo-mineral layer.

The aragonitic organo-mineral layer can be crushed in a FRITSCH Pulverisette 1 Premium Line zirconium oxide jaw and wall grinder until a crushed aragonitic powder with a grain size of 10 µm to 2 mm is obtained.

This crushed aragonitic powder can then be ground by planetary grinding. Planetary grinding can be carried out using a zirconium bowl and zirconium balls. For example, 25 zirconium balls of 20 mm diameter and 300 g of crushed aragonitic powder are placed in two zirconium bowls of 500 mL capacity each, previously frozen for 24 hours at minus 30° C. The bowls are then introduced into the grinding chamber of a FRITSCH Pulverisette 5 PL type planetary grinder for 2 grinding cycles, at a speed of 400 rpm, of 5 minutes each.

In order to optimise grinding and to prevent the powder from clogging on the bowl walls and ball surfaces, the second grinding cycle can be carried out wet, for example by adding additives in liquid form, with a high boiling point and low vapour pressure, for example water for injection (WFI) or alcohols such as isopropanol or ethanol.

WFI, refrigerated between 2 and 4° C., may be added to each bowl until a colloidal solution with a viscosity of 3.5 MPa is obtained. At the end of the second grinding cycle, an aragonitic powder with a particle size between 50 nm and 300 µm can be obtained and set aside.

These operations allow, in particular the separation and fracturing of biocrystals from the mineral fraction of the aragonitic organo-mineral layer.

The milling product of the calcitic organo-mineral layer may undergo the same grinding step as that undergone by the aragonitic organo-mineral layer, at the end of which a calcitic powder with a particle size between 50 nm and 300 µm may also be obtained.

The aragonitic and calcitic powders obtained by grinding can be sterilised with gamma radiation at 25 kGy before undergoing a hot percolation step.

### Hot Percolation Step (b)

Hot percolation is a filtration process through a permeable medium that allows wet extraction of soluble components.

Hot percolation proved advantageous for two reasons. On the one hand, optical microscopic observation of the aragonitic powder after grinding showed agglomerates of grains of different diameters stuck together by organic residues. On the other hand, hot percolation, for example in the presence of methanol, makes it possible to solubilise the protein-bound lipids of the organic fraction of the aragonitic organo-mineral layer.

This phenomenon can be explained by the structure and the physico-chemical nature of the constituents of the aragonitic organo-mineral layer.

Solubility tests have shown that these organic residues with adhesive properties are composed of the soluble and insoluble intracrystalline and interlamellar organic components of the aragonitic organo-mineral layer and the calcitic organo-mineral layer. Hot percolation allows the washing of the aragonitic powder grains which, on microscopic observation of the aragonitic percolation powder, regained their shiny appearance. Percolation can be carried out by wet sieving.

Wet sieving can be carried out with a Filtra type sieve shaker, which comprises, from top to bottom:
a cover fitted with an opening to receive a pipe for the water inlet,
6 sieves with top to bottom pore diameters: 315, 250, 125, 45, 20 and 10 µm,
a collection bottom fitted with a pipe to collect the water from the percolation.

The parameters of the sieve shaker are set to the maximum amplitude, the vibration time for a duration of about 5 minutes.

A defined amount of aragonitic powder, from 500 g to 1 kg, can be placed in the upper sieve so as to make a permeable filter of variable thickness; a tank overhanging the sieve shaker is filled with WFI at 45° C. to which 5% methanol is added in order to solubilise the lipids. According to another embodiment, a solution of urea, as a chaotropic agent, at a concentration of 4 mol/L, can be added to the WFI before percolation, in order to cleave the high-molecular-weight proteins. The solution is sprayed onto the powder, which behaves like a filtration membrane, the filtering power of which is optimised by the vibrations of the sieve shaker by creating a vortex.

In the hot percolation step (b), the grains of aragonitic powder of smaller diameters may be carried by the WFI solution from the first sieve with the largest diameter to the lower sieves on which they settle according to their diameters, down to the last sieve with the smallest diameter. For example, the last sieve may have a diameter of 10 µm which retains grains with a diameter greater than 10 µm, allowing grains of 10 µm and smaller to pass through.

The percolation product is a saturated solution composed of a liquid phase and a solid phase, said liquid phase contains all or part of the water- and fat-soluble components of the aragonitic organo-mineral layer, said solid phase contains the insoluble components of the aragonitic organo-mineral layer and aragonite grains whose diameter is less than or equal to the diameter of the last sieve, in particular from 50 nm to 10 µm.

The hot percolation step (b) also produces a percolation aragonite powder containing aragonite grains whose diameter may be greater than the diameter of the last sieve, in particular greater than 10 µm.

The hot percolation step (b) can be applied in the same way to the calcitic powder resulting from the grinding step (a) of the calcitic organo-mineral layer.

### Separation Step (c)

In order to separate the liquid phase and the solid phase from the saturated solution resulting from the hot percolation step (b), a separation step (c) can be applied to the saturated solution to recover, on the one hand, the liquid phase, and, on the other hand, the solid phase. For example, this separation step (c) can be carried out by centrifugation and the recovered liquid phase is called the supernatant and the recovered solid phase is called the pellet. Only this example is described here but the skilled person will know how to implement separation techniques different from centrifugation to carry out this step (c).

The separation step (c) carried out by centrifugation may be carried out in a 2-litre Lisa-type centrifuge fitted with 4 baskets capable of accommodating 4 vials each containing 300 mL of solution. The rotation speed can be increased to 18 000 rpm, the temperature set at 5° C. and the rotation time set at 20 minutes.

The aragonitic pellet and/or the calcitic pellet may be dried, for example in an oven at 25° C. for 12 hours. The aragonitic pellet and/or the calcitic pellet may have a particle size between 10 µm and 50 nm and may contain insoluble protein and non-protein components. The aragonitic pellet may then be spheronised and set aside for sterilisation by gamma radiation at 25 kGy. The calcitic pellet may be set aside.

The centrifugal separation step (c) may be performed one or more times.

### Supercritical CO₂ Treatment Step (d)

It is known that the soluble molecules of interest are most often intracrystalline and require the dissolution by acid hydrolysis of calcium carbonate biocrystals, whether aragonitic or calcitic. For this reason, the process of the invention comprises the supercritical CO₂ treatment step (d).

It is known that CO₂ in the supercritical state has very special properties: a diffusivity coefficient, the possibility of extracting soluble components that are rather low-molecular-weight and apolar, as well as fats, without generating polluting residues. It also has disinfectant properties against viruses and bacteria. In addition, the addition of co-solvents increases its solvent power. It also has a low coefficient of viscosity and a lack of surface tension, which increases its penetration power, facilitated by the physico-chemical nature of aragonite and calcite biocrystals, hydrophilic biomaterials, permeable to gases, including CO₂, a fortiori when it is supercritical.

The installation of a reactor for treatment with supercritical CO₂ comprises 5 main elements:
an inlet and storage tank for CO₂ in the gaseous state,
a condenser for converting gaseous CO₂ into liquid CO₂,
a liquid CO₂ storage tank,
a heat exchanger for converting liquid CO₂ into supercritical CO₂,
a reactor where the extraction of soluble molecules takes place, and
one or more extractors.

The supercritical CO₂ treatment step (d) can be applied to the aragonitic percolation powder according to the following procedure: in the reactor of adequate size, connected to the supercritical CO₂ exchanger, the aragonitic percolation powder collected in the sieves after the hot percolation step (b) is placed. When the valve of the exchanger is opened to release the supercritical CO₂, the latter is injected into the reactor where the extraction reaction of the molecules of interest (soluble biopolymers, fatty acids, lipids, pigments) takes place. At the outlet, the dissolved substances are recovered according to their nature in one or two extractors connected to the reactor, where the lowering of temperature and pressure causes them to precipitate in dry form. The CO₂ becomes gaseous again upon exit, where it is recovered for use in a new extraction cycle.

The result is, on the one hand, a supercritical CO₂-treated aragonitic powder and, on the other hand, soluble components from the aragonitic organo-mineral layer, which can then be sterilised with gamma radiation at 25 kGy and set aside.

The supercritical CO₂ treatment step (d) can be applied in the same way to the calcitic percolation powder and optionally to the calcitic pellet.

### Filtration Step (e)

After the separation step (c) carried out by centrifugation, the aragonitic supernatant and/or the calcitic supernatant can be filtered in a filtration step (f) to obtain a filtered aragonitic supernatant and/or a filtered calcitic supernatant which can then be set aside.

For example, the filtration step (f) can be performed on a Celite bed or a membrane.

### Concentration Step (f)

The filtered aragonitic supernatant and/or the filtered calcitic supernatant can be concentrated, for example, with the Buchi type Rotavapor at a temperature of 40° C., a heating flask rotational speed of 10 rpm and a vacuum of 23.33 mbar.

This concentration step (f) produces an aragonitic concentrate and/or a calcitic concentrate with a concentration factor of up to ¼. This concentrate can have an allochromatic colouring varying from yellow to orange, from red to brown or grey, colours due to the presence of pigments coming from the metals contained therein, i.e. Mn, Fe, Zn, Ba, Sr, Mg, Cu, Al, Ni, V, Cr, Mo.

### Sonication Step (g)

Advantageously, it is possible to modify and optimise the physico-chemical properties of the aragonitic concentrate and/or the calcitic concentrate resulting from the concentration step (f) by subjecting it to a sonication step (g) by sonochemistry.

Sonication is a process using mechanical and acoustic waves in a liquid medium, using for example a sonotrode, at a frequency between 20 kHz and 200 kHz depending on the initial viscosity of the concentrate. Advantageously, sonication makes it possible to trigger and accelerate reactions and thus to modify and potentiate the pharmacological and pharmacodynamic properties of active soluble molecules.

Indeed, cavitation causes the formation of highly reactive hydroxylated radicals, which results in an improvement in the yield of the reactions, a decrease in the reaction time of the molecules of interest with each other and an exponential potentiation of the antiradical properties of some of them.

The solution to be treated can be placed in an ultrasonic tank in which a sonotrode is immersed with its tip at least 1 cm from the surface and the walls, in order to avoid the formation of electric arcs.

The sonication step (g) may be applied for 30 min, after which an increase in the viscosity of the concentrate may be observed. The concentrate is then in the form of a stable colloidal emulsion thanks to the physico-chemical modification and the arrangement of the collagen components. The colloidal emulsion can then be sterilised either by microfiltration, sterilising filtration or 25 kGy gamma radiation. The product can be kept at a temperature of 5° C.

### Cold Acid Hydrolysis Step (h) and Washing and Supercentrifugation Step (i)

In order to collect biopolymers and other insoluble components contained in the supercritical CO₂-treated aragonitic powder and/or supercritical CO₂-treated calcitic powder, said powder may then be subjected to cold acid hydrolysis.

The supercritical CO₂-treated aragonitic powder and the supercritical CO₂-treated calcitic powder may be combined and placed in a refrigerated hydrolysis reactor of adequate capacity filled with pyrogen-free water at 2° C. An adjustment of the ionic strength can be made beforehand in order to weaken possible ionic, mineral matrix/protein interactions. NaCl is added to the solution at 0.5 mol with stirring for 30 min, i.e., depending on the ratio, 1 kg of powder to 25 litres of water and 5 litres of NaCl. A first centrifugation is then carried out at 18 000 g, for example. The pellet is taken up in an adequate amount of pyrogen-free water to which 80% acetic acid is added in the same ratio. The whole is maintained at a temperature between 1 and 4° C. for a pH below 4.5, under constant stirring. An emulsion is obtained which is diluted with pyrogen-free water to break it; the presence or absence of undissolved calcium carbonate is checked with oxalic acid. This is eliminated through a gauze and by decantation. At this step, a suspension of insoluble proteins and other components, including insoluble pigments, is obtained, which is continuously centrifuged, for example, at 18 000 g. The centrifugation pellet is taken up again under stirring in the same amount of 5% diluted acetic acid, intended to dissolve any calcium carbonate residue. The centrifugation pellet is washed twice in the same amount of non-pyrogenic water and the pH is adjusted to 7 by the addition of sodium hydroxide solution.

Each wash is followed by supercentrifugation, at the end of which a wet paste of insoluble proteins is obtained, which is dried either by freeze-drying or by spraying. The dry product obtained is ground to a greyish powder with a particle size between 10 µm and 50 nm, which is sterilised with gamma radiation at 25 kGy and set aside.

At the end of these different steps it is possible to obtain:
a sterilised powder of spheronised aragonitic grains, with a particle size between 50 nm and 10 µm originating from the pellets recovered during the separation step (c) carried out by centrifugation after the hot percolation step (b) of the aragonitic powder,
a colloidal emulsion originating from the sonication step (g) composed of soluble polymers, soluble organic pigments (beta-carotene), metals, metalloproteins, metalloenzymes, growth factors, glycoproteins, glycosamines, polyunsaturated lipids and fatty acids,
biopolymers and soluble organic pigments from the supercritical CO₂ treatment step (d), and
insoluble, so-called support and structural biopolymers, insoluble pigments recovered from the hydrolysis (h) and washing and supercentrifugation (i) steps.

All these extracts are intended to be used in whole or in part in the formulation of medical devices, preparations for therapeutic purposes, for use in orthopaedic surgery, minimally invasive surgery, dermatology, stomatology, maxillofacial surgery, aesthetic medicine and in the formulation of dermocosmetic products.

The following non-limiting examples illustrate the applications of the invention as described above.

### EXAMPLES

### Example 1: Formulation of a Sealing Cement for Use in Arthroplasty

It is known that osteoarthritis, of the hip, shoulder, knee or any other joint, is the most frequent joint pathology due to the combined action of ageing and joint constraints.

This is because the cartilage coating on the joint surfaces wears away and the progressive deterioration leads to changes that result in pain and functional impotence, such as those that occur in hip disease, which eventually require a prosthesis. The prosthesis generally consists of a hemispherical cup implanted in the acetabulum of the iliac bone, a stem implanted in the femoral shaft and ending in a hemispherical head. These two parts are articulated together by means of a polyethylene or ceramic insert. The stem of the femoral prosthesis and the cup are usually either sealed with a methyl methacrylate-based surgical cement or impacted.

Given the post-operative complications sometimes encountered when using methyl methacrylate-based surgical cement (setting reaction with temperature rise to over 70° C.), ageing and shrinkage of the cement, complications which generally end with the prosthesis being resealed or sometimes impacted, without cement; the aim being to achieve a physiological mechanical anchorage by bone regrowth around the implant, itself sometimes covered with a synthetic biomaterial favouring the regrowth process.

For this reason, this example is a sealing cement with the following centesimal formulation:
95 g aragonitic powder obtained in the separation step (c) carried out by centrifugation and spheronised (particle size between 50 nm and 10 µm),
4 g carbonated calcium carbonate,
1 g sodium hydrogen phosphate,
90 mL colloidal emulsion obtained in the sonication step (g),
5 g carboxylmethyl cellulose sodium

The use of spheronised aragonitic powder, with a particle size between 50 nm and 10 µm, is justified for the following reasons: spheronisation is initially intended to ensure better injectability and flowability of the cement and to promote the creation of interconnected porosity with pores of 10 to 100 µm essential for osteo-conduction.

It is known that a cement sealant, once dry, must have a compressive strength at least equal to that of the receiving bone. Knowing that the Young’s modulus in compression of the aragonitic organo-mineral layer is 141 MPa and that that of the cortical bone is 131 MPa, it is therefore understandable that the cement is perfectly adapted to provide better anchorage and load distribution, as well as better resistance than conventional cements.

The presence of carbonated calcium carbonate in the cement formulation is justified because of the plasticity, adhesion and cohesion properties acquired by calcium carbonate during the carbonation process.

Soluble and insoluble proteins, “signal” molecules that stimulate cell differentiation and proliferation as well as osteogenesis, play a key role in building bone architecture. The addition of carbonated calcium carbonate confers plasticity, adhesiveness and malleability to the whole, favoured by the spheronisation of aragonitic grains, making handling and insertion easier.

The addition of a setting accelerator makes it possible to modify the preparation time, the initial setting time and the final setting time. The colloidal emulsion obtained in the sonication step (g), added to the mixture, allows a fluid, homogeneous and stable paste to be obtained.

The cement was used to experimentally seal a stent stem in the femoral shaft of a calf. The postoperative X-ray showed a densification around the prosthesis stem, characteristic of the presence of calcium carbonate, the major component of the mineral fraction of the inner aragonitic organo-mineral layer, a densification that will progressively decrease during the transformation of the cement into new bone and merge with that of the recipient bone. The prosthesis then behaves like an impacted prosthesis.

Biopsies taken at 4 months showed the presence of newly formed bone around the prosthesis stem, without thinning of the cortex, signalling the transformation of the cement into autologous cancellous bone and cortical bone.

These findings can be explained by the presence, among others, of low-molecular-weight glycoproteins with “BMP2-like” effects and their osteo-inductive properties, aminoglycosides with antibiomimetic properties, pigments, amino acids and proteins including glycosaminoglycans.

### Example 2: Shapeable Bone Substitute for the Revision of Osteoarticular Prostheses

It is known that during a hip, shoulder or knee prosthesis revision, the removal of residual methyl methacrylate cement can cause significant decay of the bone structures due to the need to perform approaches to remove all residual cement. Reconstructive osteosynthesis uses either autologous grafts or the use of a synthetic bone substitute, which does not preclude the use of a surgical cement to seal the prosthesis.

This is why this example is a bone substitute that is used not only to fill in the substance losses caused by the need to perform the access procedure, but also to seal the new prosthesis. The formulation of the substitute of the example is as follows:
85 g aragonitic powder obtained in the separation step (c) by centrifugation and spheronised (particle size between 10 µm and 200 µm),
2.5 g soluble biopolymers obtained in the supercritical CO₂ treatment step (d),
2.5 g insoluble biopolymers obtained in the steps of cold acid hydrolysis (h) and washing and supercentrifugation (i),
5 g carbonated calcium carbonate,
75 mL colloidal emulsion obtained in the sonication step (g),
5 g sodium hydrogen phosphate.

The granulometry of the aragonitic powder is chosen to favour the creation of an open and interconnected porosity, conducive to rapid osteo-conduction, associated with the osteo-inductive properties of the bone substitute, and also with its antibiomimetic properties.

Various uses of the bone substitute of the example are presented.

Inpatient recovery of a critical clinical case, after failure to reduce humerus fracture after rupture of the medullary nail with staphylococcal sepsis, fistulated at the elbow.

The bone substitute was used, after removal of the fractured orthopaedic material, trimming of the necrotic tissue and placement of an osteosynthesis plate, without antibiotic prophylaxis.

The antibiomimetic properties have been confirmed by microbial load tests on the product according to the invention before use, which have shown an inhibition of microbial proliferation, particularly on strains of Candida albicans, Aspergillus brasiliensis, Staphylococcus aureus, Pseudomonas aeruginosa, Bacillus subtilis.

The postoperative follow-up showed sedation of the infectious episode and the postoperative X-rays at 3 months showed a restitution ad integrum of the bone tissue.

The bone substitute was also used in the hospital setting in another critical clinical case of resumption of treatment of a comminuted small fragment fracture of the lower third of the femur, 15 cm long, after failure of orthopaedic treatment with a medullary nail and two attempts at iliac grafts over a period of 2 years, with the following clinical picture: rhabdomyolysis, coma and life support. After the placement of an external fixator, removal of orthopaedic material and bone sequestration, the exemplified bone substitute was shaped into a cylinder with the dimensions of the loss of substance and placed between the distal and proximal fragments. Postoperatively at 4 months allowed unipodal support and near-normal gait at 7 months. Radiological control showed not only reconstruction of the cortical bone of normal thickness, but also permeabilization of the medullary canal between the proximal and distal fragments of the restored femur.

All these observations highlighted the osteo-inductive properties of the bone substitute, as well as its osteo-conductive and antibiomimetic properties, and its ability to be dependent on the recipient’s local systemic regulation.

The inventors also propose the use of the bone substitute in minimally invasive surgery, in kyphoplasty, in vertebroplasty, in the treatment of osteoporosis, fractures and vertebral compression.

Clinically, rapid hardening of the bone substitute is observed at a body temperature of 37° C. despite the humidity of the surgical site.

On the other hand, the cohesion, plasticity and adhesive properties of the bone substitute considerably limit the possibility of vascular leakage due to demixing.

The bone substitute of the example can also be used in maxillofacial surgery and stomatology.

### Example 3: Topical Preparation for the Treatment of Severe Refractory Dermatoses

It is known that certain forms of dermatoses such as psoriasis of the elbows and scalp, which develop in patches, are sometimes refractory to any treatment, including topical dermocorticoids. This is why the inventors are proposing a preparation adapted to plaque psoriasis. Indeed, the acceleration of keratogenesis produces an anarchic thickening of the stratum corneum of the skin, leading to the formation of hyperplastic keratin plaques that prevent topical penetration.

The formulation of the topical preparation of the example is:
20 g aragonitic powder obtained in the separation step (c) carried out by centrifugation and spheronised (particle size 50 nm to 10 µm),
2 g soluble biopolymers obtained in the supercritical CO₂ treatment step (d),
5 g urea,
10 g allantoin,
3 g salicylic acid,
60 mL colloidal emulsion obtained in the sonication step (g),
1 mL mixture of essential and vegetable oils
Excipients W/O q.s. 100 g

The formulation of the mixture of essential and vegetable oils used in the topical preparation of the example is as follows:
1 mL Lavandula angustifolia
1 mL Chamaemelum nobile
1 mL Melaleuca alternifolia
1.5 mL Helychrisum italicum
1 mL Juniperus oxycedrus
1.5 mL Myrtus communis
20 mL Argania spinosa
50 mL Persea Americana
10 mL Borago officinalis,
13 mL Wheat germ

In practice, a solution containing 20 g of aragonitic powder, 2 g of soluble biopolymers, 30 mL of colloidal emulsion is prepared until a gel is obtained which is kept at a temperature between 2 ° and 5° C.

A mixture containing 5 g of urea, 10 g of allantoin, 3 g of salicylic acid and 30 mL of colloidal emulsion is also prepared.

The whole is mixed for 30 minutes until completely solubilised, then placed in an oven at 25° C. for 24 hours and stirred every 6 hours to control the release of CO₂. All the components are then placed in a blender to be mixed for 1 hour.

### Example 4: Topical Preparation for the Treatment of Vitiligo

Vitiligo is known to be a non-contagious and serious dermatosis that is difficult and time-consuming to treat, with very significant psycho-social repercussions, which affects 0.5 to 2% of the world’s population and whose progression is unpredictable. It causes depigmentation of the skin, either by diffuse patches, by areas or generalised. It is manifested by the appearance of white patches due to the disappearance of melanocytes, the cells that produce melanin, the main pigment of the skin.

Therapeutic possibilities are limited. They range from the use of UVB, to dermocorticoids and biosimilars, topical preparations and, as a last resort, to surgical grafts of melanocytes or thin skin grafts. To date, there is no effective universal treatment for vitiligo. In addition, most of the proposed treatments may have embarrassing or serious side effects. In addition, vitiligo is often accompanied by an alteration and thinning of the skin’s surface, due to a fragility of the keratinocytes which are accidentally eliminated by microtrauma in the areas of friction. There is also the disappearance of melanocytes and hair bulbs, reservoirs of melanin, due to dysfunction in their maturation, due to a problem of cohesion and attachment with the basement membrane and adjacent keratinocytes.

Taking into account the physiopathology of vitiligo, the inventors propose a topical preparation intended to modify the metabolism of the dermocutaneous zone by inducing the maturation, recruitment, proliferation and differentiation of stem cells of all types, in particular melanocytes, keratinocytes and fibroblasts.

These local metabolic inductions are also manifested by a progressive recoloration of the skin’s surface, resulting from capillary angiogenesis.

The formulation of the topical preparation of the example is as follows:
20 g aragonitic powder obtained in the separation step (c) carried out by centrifugation and spheronised (particle size between 50 nm and 10 µm),
2 g soluble biopolymers obtained in the supercritical CO₂ treatment step (d),
5 g insoluble biopolymers obtained in the steps of cold acid hydrolysis (h) and washing and supercentrifugation (i),
40 mL colloidal emulsion obtained in the sonication step (g),
0.5 mL blend of essential and vegetable oils,
5 g urea
Excipient O/W q.s. 100 g

In practice, 20 g of spheronised aragonitic powder, 2 g of soluble biopolymers, 5 g of insoluble biopolymers, 30 mL of colloidal emulsion are mixed until a gel is obtained which is kept at a temperature between 2° C. and 5° C.

5 g of urea and 10 mL of colloidal emulsion are also prepared, which are mixed for 30 minutes until completely solubilised.

All the components are then mixed in a blender for 1 hour: the resulting preparation is placed in an oven at a temperature of 25° C. for 24 hours, with stirring every 6 hours to control the release of CO₂.

The components of the topical preparation are, among others, low-molecular-weight glycoproteins with BMP-like properties, including TNFβ, EGF, TGFβ, which have biological activities in the synthesis, proliferation, maturation of all cell lines of the basal layer of the epidermis and especially on melanocytes. It also naturally contains free pigments such as beta-carotene, precursor of vitamin A, which plays an essential role in the synthesis of melanin; also melanic pigments associated with porphyrins, with enzymes, in the form of metalloporphyrins, metalloenzymes, which are involved in the colouring of biological tissues.

On the other hand, the mixture of essential and vegetable oils of the topical preparation of the example has the following formulation per 100 mL:
Evening primrose 45 mL
Wheat germ 50 mL,
Piperine 1 mL,
Helycrisum italicum 1 mL,
Melaleuca alternifolia 1 mL
Lavandula angustifolia 1 mL,
Salvia oficinalis 1 mL

These extracts are intended to potentiate all the properties of the components of the topical according to the invention.

The aragonitic powder is observed to contain almost all the amino acids including tyrosine and cysteine essential for melanin synthesis. All these elements associated with ultra-pure calcium, play a fundamental role in reducing inflammation, strengthening the local immune system, in the recoloration of the teguments and in the bioavailability of constituents.

The pharmacological properties and the interactions of the natural components of the topical preparation also have an action on the stimulation and multiplication of the melanosomes as well as on the transfer of the matrix melanin present in the melanosomes to the surrounding keratinocytes, which ensure the turnover of the epidermal population as well as the regeneration of the hair follicles, reservoirs of melanin.

On the other hand, it is known that the soluble molecules of interest contained in the colloidal emulsion after sonication, such as low-molecular-weight proteins related to growth factors or cytokines, which also have pleiotropic properties, inhibit lipid peroxidation by preventing the binding of singlet oxygen (¹O₂) to the double bonds of polyunsaturated fatty acids. This has the effect of preventing the deterioration of these acids, proteins and biomolecules in general, a deterioration responsible for the production of new free radicals harmful to the skin’s surface.

The topical preparation is recommended in the treatment of vitiligo naïve of any previous treatment; however, in the case of old vitiligo or those which have been subjected to iterative treatments without visible results, which generally cause the migration of melanocytes and keratinocytes as well as the disappearance of hair bulbs, it is possible, with the aim of provoking a more active and deeper penetration of the topical according to the invention, up to the basal layer, to combine the application of the latter with the use of a medical iontophoresis device, the principle of which is to promote the transcutaneous penetration of an ionisable product by the application to the skin of a galvanic current of low intensity by means of an electrode causing the migration of the ions in the chosen direction according to the polarity of the electrode.

### Example 5: Cosmetic Preparation for Correcting Wrinkles and Dermal Depressions

The inventors propose the cosmetic use of the composition according to the invention for correcting ptosis, dermocutaneous depressions, deep and superficial wrinkles, for preventing body ageing.

The formulation of the composition of the example is as follows:
29 g aragonitic powder obtained in the separation step (c) carried out by centrifugation and spheronised (particle size 10 to 45 µm)
1 g soluble biopolymers obtained in the supercritical CO₂ treatment step (d), 70 mL of carboxylmethyl cellulose sodium gel, itself composed of:
   68 mL of colloidal emulsion obtained in the sonication step (g), and
   2 g of carboxylmethyl cellulose sodium.

In practice, a solution of carboxylmethyl cellulose sodium is first prepared: 68 mL of colloidal emulsion, 2 g of carboxylmethyl cellulose sodium is put in a mixer. The mixture is stirred for 20 minutes and left in the cold at 5° C. for 12 hours until a gel is formed.

At the end of this period, 29 g of spheronised aragonitic powder and 1 g of soluble biopolymers are then mixed with the 70 mL of gel previously obtained.

The composition is packaged in 1 mL syringes, provided with 0.4 mm/20 mm screwed needles, which are then double wrapped and sterilised with gamma radiation at 25 kGy.

The injection of the composition in deep or superficial wrinkles, in dermocutaneous ptosis, induces, in addition to its volumising properties, a stimulation of the maturation and proliferation of fibroblasts producing type I collagen responsible for the tonicity, suppleness and firmness of the skin.

The composition has significant advantages due to its physico-chemical composition, the properties of its natural components which result in the absence of painful and inflammatory postoperative phenomena. Moreover, the exemplified composition is dependent on the local systemic regulation of the recipient and produces its corrective effects for a prolonged period of time.

### Example 6: Protective, Tan-Accelerating Dermocosmetic Preparation

Tanning is the skin’s defence and adaptation reaction to damage by the sun and more precisely by UVA and UVB rays, by colouring the skin through an increased production of melanin by the melanocytes: this is tanning. Immoderate exposure to the sun causes the system to race out of control, and the resulting oxidative stress induces sunburn, allergies, pigmentation spots, burns, skin ageing, not forgetting the fact that repeated over-exposure eventually causes an alteration of the cells’ micro RNA which can lead to their deterioration and the appearance of skin cancers.

For this reason, protective products have been developed that contain several types of components that help melanocytes fight oxidative stress and produce more melanins. Each individual produces more or less melanins; these are unevenly distributed across races and skin types. There are two kinds of melanins produced by melanocytes, black-coloured eumelanins and red-and-yellow-coloured pheomelanins: eumelanins are more resistant to sun damage and are produced in greater amounts by individuals with black or brown skin; pheomelanins predominate in individuals with light or reddish skin. They are more rapidly altered by sun damage and protect the skin less from oxidative stress caused by the sun.

In the description of the process according to the invention, the inventors highlighted the presence of polyunsaturated fatty acids, tyrosine and cystine which promote the synthesis of melanins, metalloenzymes which play a fundamental role in skin colouring, cytokines which strengthen the local immune system, and matrix melanins produced during the stimulation of melanocytes.

For this reason, the composition of this example for the preparation of a sunscreen has the following formulation:
10 g aragonitic powder obtained in the separation step (c) carried out by centrifugation and spheronised (particle size between 50 nm and 10 µm),
5 g insoluble biopolymers obtained in the steps of cold acid hydrolysis (h) and washing and supercentrifugation (i),
1 g soluble biopolymers obtained in the supercritical CO₂ treatment step (d),
20 mL colloidal emulsion obtained in the sonication step (g),
10 mL concentrated Coco Nucifera solution
0.05 mL Ascorbyl palmitate,
Excipient q.s. 100 mL

In practice, 10 g of spheronised aragonitic powder, 5 g of insoluble biopolymers, 1 g of soluble biopolymers, 20 mL of colloidal emulsion, 10 mL of concentrated Coco Nucifera solution, 0.05 mL of ascorbyl palmitate are prepared. The mixture is placed in a blender and mixed for 1 hour. The excipient is then added to the mixture: the whole is mixed for 1 hour until a cream is obtained.

The protection factor found under experimental conditions is between 10 and 40.

The composition was tested on a dozen light-skinned individuals ranging from redheads to blondes, whose sun exposure always resulted in erythema, even burns and a lack of tanning. The application of the composition of the example over a period of 10 days with summer sunshine enabled all the individuals tested not only to avoid the occurrence of erythema or burns but also to ensure a uniform tan resulting from a stimulation of melanogenesis.

The steps of the process of the invention have made it possible to obtain the totality of the active molecules contained in the inner aragonitic and outer calcitic organo-mineral layers of the shells of the molluscs mentioned in reference.

### Example 7: Production of an Injectable Solution for Use in Biotherapy

It is known from the study of anatomy, pathophysiology, reproduction and interaction with their biotope that the bivalve molluscs cited in the present patent construct their shells by synthesising the organic and inorganic components that are excreted in the extra-pallial cavity to form the extra-pallial fluid according to two sequential processes: a first cellular process comprising ion transport, glycoprotein synthesis and a second physicochemical process.

As a result of these processes, in the inner aragonitic and outer calcitic layers are found all the active molecules described in the present patent; thus, in the inner aragonitic layer are found growth factors, low-molecular-weight glycoproteins (8 to 50 kDa), interleukins, chemokines, TNFs (a group derived from a common ancestral gene), TGFs, prostaglandins, etc.

Pre-clinical and clinical observations seem to demonstrate that cytokines contained in the organic fractions of the aragonitic and calcitic organo-mineral layers of the mentioned molluscs have a paracrine mode of action by becoming locally systemically dependent on the recipient host. These cytokines, which originate from the metabolic activities of the immune defences of the mentioned bivalve molluscs, are found in the extra-pallial fluid and are part of the soluble molecules of the inner aragonitic and outer calcitic layers of these molluscs.

This is why the inventors propose the use of an injectable solution that can be used in biotherapy, particularly in certain chronic auto-inflammatory pathologies such as rheumatoid polyarthritis, Crohn’s disease, arteriosclerosis, type II diabetes, ankylosing spondylitis, ulcerative colitis, severe psoriasis and psoriatic arthritis.

It is known that psoriasis results from the interaction between keratinocytes, dendritic cells, and T-lymphocytes that activate each other. The inflammatory cytokines produced by these 3 cell types, TNFα, IL-23 and IL-17, are preponderant in this pathology, due to the cytokine storm they produce. Immunobiological treatments have the property of blocking the effects of these 3 cytokines whose activation favours the appearance and perpetuation of psoriasis, the aetiology of the latter remaining multifactorial. These therapeutics block the action of the cytokines thanks to anti-cytokine monoclonal antibodies which aim to inhibit the functions of the dendritic cells by preventing the production of IL-23, as well as the interleukins IL-17 and IL-22.

Biologic biotherapy agents in the treatment of inflammatory diseases have proven to be effective but are not without adverse effects as shown by a recent meta-analysis that reports reactivation of latent tuberculosis and lymphoma, atypical and opportunistic infections, viral infections (shingles), hepatitis B or C, demyelinating, neoplastic, cardiovascular, hepatotoxicity, cytopenia, hypercholesterolemia, as well as injection site reactions and pulmonary and digestive complications.

The hot percolation, centrifugation, concentration, sonication and supercritical CO₂ treatment steps allow the extraction of active molecules such as cytokines and growth factors. These molecules have pleiotropic properties that determine several phenotypic traits and local systemic activity, participating in tissue homeostasis regulation, particularly anti-inflammatory, on cytokines of inflammation such as TNFα, interleukins IL-23, IL-17, common to many pathologies.

The intramuscular, intravenous and/or subcutaneous injection biotherapy solution in this example can be formulated as follows:
2 g soluble biopolymers obtained in the supercritical CO₂ treatment step (d),
Colloidal emulsion obtained in the sonication step (g) q.s. 100 mL

In practice, soluble biopolymers are added to the colloidal emulsion and then placed in an oven at 25° C. for 12 h, with stirring every 6 h until completely dissolved. The whole is then packaged in crimped ampoules which are sterilised with gamma radiation at 25 kGy.

### Example 8: Culture Medium for Maturation and Proliferation of Animal and Human Stem Cells and Progenitor Cells

It is known that the building, growth and repair of tissues, organs and systems depend on the action of different growth factors or cytokines, alerted first by stem cell markers and then by those of progenitor cells.

It is also known that multipotent stem cells give rise to several cell lines for different tissues, organs and systems. Progenitor cells are an advanced step of stem cells, albeit with limited dividing properties; they are the basis of tissue repair and, due to their reduced mobility, are found in close proximity to target tissues.

In vitro studies have highlighted the potentiating action of soluble molecules contained in the organic fractions of the inner aragonitic and outer calcitic layers of the molluscs mentioned.

The inventors therefore also propose the preparation of culture media for the maturation and proliferation of animal and human stem cells and progenitor cells.

Such a medium includes:
5 g aragonitic powder obtained in the separation step (c) carried out by centrifugation and spheronised (particle size between 50 nm and 10 µm),
2 g soluble biopolymers obtained in the supercritical CO₂ treatment step (d),
0.4 g glucose,
2 mL autologous human serum, and
Colloidal emulsion obtained in the sonication step (g): q.s. 100 g.

In practice, such a culture medium is placed in a bioreactor, which may be aerobic or anaerobic, into which autologous progenitor cells, muscle or periosteal progenitor cells are introduced for multiplication, for an incubation period of 0 to 15 days.

In practice, progenitor cells are obtained by biopsies, extracted by enzymatic digestion in the usual way. After incubation, the preparation can be used in the donor subject for indications such as tissue regeneration of all types: severe burns, extensive wounds, muscle destruction, extensive loss of substance, periodontal diseases. It can be used during routine surgery, either by injection or in minimally invasive surgery.

### Example 9: Production of a Capsule for “Per Os” Administration

With the aim of continuing by “per os” administration the treatment by injection of the above-mentioned pathologies, the inventors propose the following composition:
60 g spheronised aragonite powder,
1 g soluble biopolymers,
2 g insoluble biopolymers,
10 g acerola powder, and
Colloidal emulsion q.s. 100 g.

In practice, the aragonite powder, the soluble and insoluble biopolymers and the acerola powder are mixed with the colloidal emulsion. The mixture is mixed for 10 minutes and then placed in an oven at 30° C. for 3 hours until a paste with a viscosity of 10² Pa·s. is obtained.

The whole is packaged in acid-resistant vegetable capsules with a capacity of 0.8 mL with delayed dissolution.

1-13. (canceled)

14. A process for isolating molecules contained in an aragonitic organo-mineral layer and/or in a calcitic organo-mineral layer of a shell of a marine bivalve mollusc comprising the following steps:
(a) grinding the aragonitic organo-mineral layer and/or the calcitic organo-mineral layer to obtain an aragonitic powder and/or a calcitic powder;
(b) hot percolating the aragonitic powder and/or the calcitic powder to obtain:
on the one hand, a saturated aragonitic solution and/or a saturated calcitic solution, said saturated aragonitic solution comprising an aragonitic liquid phase and an aragonitic solid phase and said saturated calcitic solution comprising a calcitic liquid phase and a calcitic solid phase, and
on the other hand, an aragonitic percolation powder and/or a calcitic percolation powder;
(c) separating the saturated aragonitic solution and/or the saturated calcitic solution to recover:
on the one hand, the aragonitic liquid phase and/or the calcitic liquid phase, and
on the other hand, the aragonitic solid phase and/or the calcitic solid phase; and
(d) treating the aragonitic percolation powder and/or the calcitic percolation powder with supercritical CO₂ to obtain:
on the one hand, a supercritical CO₂-treated aragonitic powder and/or a supercritical CO₂-treated calcitic powder, and
on the other hand, all or part of the soluble molecules contained in the aragonitic organo-mineral layer and/or in the calcitic organo-mineral layer.

15. The process according to claim 14, wherein the marine bivalve mollusc is selected from Pinctada Maxima, Pinctada Margaritifera, Pinctada Martensi, Pinctada Fucata, Tridacnae Gigas, Tridacnae Maxima, Tridacnae Hippopus Hippopus, Tridacnae Derasa, Tridacnae Tevaroa, Tridacnae Crocea, Tridacnae Squamosa, Tridacnae Porcelanus and mixtures thereof.

16. The process according to claim 14, wherein the hot percolation step (b) is carried out by wet sieving with a liquid whose temperature is above 30° C.

17. The process according to claim 16, wherein the liquid used in the hot percolation step (b) is an aqueous solution, in particular an aqueous solution comprising methanol, an aqueous solution comprising a urea solution or a mixture thereof.

18. The process according to claim 14, wherein the separation step (c) is carried out by centrifugation and the recovered liquid phase is referred to as supernatant and the recovered solid phase is referred to as pellet.

19. The process according to according to claim 18, comprising, after the separation step (c) carried out by centrifugation, the following steps:
(e) filtering the aragonitic supernatant and/or the calcitic supernatant to obtain a filtered aragonitic supernatant and/or a filtered calcitic supernatant;
(f) concentrating the filtered aragonitic supernatant and/or the filtered calcitic supernatant to obtain an aragonitic concentrate and/or a calcitic concentrate;
(g) sonicating the aragonitic concentrate and/or the calcitic concentrate to obtain an aragonitic colloidal emulsion and/or a calcitic colloidal emulsion.

20. The process according to claim 14, further comprising, after the supercritical CO₂ treatment step (d), the following steps:
(h) cold acid hydrolysis of the supercritical CO₂-treated aragonitic powder and/or supercritical CO₂-treated calcitic powder to extract the insoluble molecules present in said powders; and
(i) washing and supercentrifugation to isolate and recover said insoluble molecules.

21. A composition comprising:
the aragonitic solid phase and/or the calcitic solid phase recovered during the separation step (c) as defined in said process according to claim 14, and at least one component selected from:
the soluble molecules obtained in the supercritical CO₂ treatment step (d) as defined in said process,
an aragonitic colloidal emulsion and/or the calcitic colloidal emulsion obtained, after said separation step (c) carried out by centrifugation, and from the following steps:
(e) filtering the aragonitic supernatant and/or the calcitic supernatant to obtain a filtered aragonitic supernatant and/or a filtered calcitic supernatant;
(f) concentrating the filtered aragonitic supernatant and/or the filtered calcitic supernatant to obtain an aragonitic concentrate and/or a calcitic concentrate;
(g) sonicating the aragonitic concentrate and/or the calcitic concentrate to obtain an aragonitic colloidal emulsion and/or a calcitic colloidal emulsion, and
insoluble molecules recovered, after the supercritical CO₂ treatment step (d) of said process, and from the steps of:
(h) cold acid hydrolysis of the supercritical CO₂-treated aragonitic powder and/or supercritical CO₂-treated calcitic powder to extract the insoluble molecules present in said powders; and
(i) washing and supercentrifugation to isolate and recover said insoluble molecules.

22. A medicinal product comprising the composition according to claim 21.

23. A culture medium, in particular as culture medium for the maturation and/or proliferation of stem cells or progenitor cells, comprising the composition according to claim 21.

24. A cosmetic composition for the correction of ptosis, dermocutaneous depressions, deep and superficial wrinkles, and/or for the prevention of body ageing, comprising the composition of claim 21.

25. A composition comprising:
the soluble molecules obtained in the supercritical CO₂ treatment step (d) of said process as defined in claim 14, and
an aragonitic colloidal emulsion and/or the calcitic colloidal emulsion obtained, after the separation step (c) of said process carried out by centrifugation, and from the following steps:
(e) filtering the aragonitic supernatant and/or the calcitic supernatant to obtain a filtered aragonitic supernatant and/or a filtered calcitic supernatant;
(f) concentrating the filtered aragonitic supernatant and/or the filtered calcitic supernatant to obtain an aragonitic concentrate and/or a calcitic concentrate;
(g) sonicating the aragonitic concentrate and/or the calcitic concentrate to obtain an aragonitic colloidal emulsion and/or a calcitic colloidal emulsion.

26. A medicinal product comprising the composition according to claim 25.
